(19) United States
(12) Patent Application Publication
(10) Pub. No.: US 2024/0009408 A1
(43) Pub. Date: Jan. 11, 2024

(54) DRUG DELIVERY HUB FOR VENTILATION MASK

(71) Applicant: SunMed Group Holdings, LLC, Grand Rapids, MI (US)

(72) Inventors: Christopher M. VARGA, Laguna Hills, CA (US); Ryan G. REDFORD, Irvine, CA (US); Thomas DILLINGHAM, Aliso Viejo, CA (US); Brian W. PIERRO, Yorba Linda, CA (US)

(21) Appl. No.: 17/798,054

(22) PCT Filed: Jan. 29, 2021
(86) PCT No.: PCT/US2021/015868

§ 371 Date: Aug. 5, 2022

Related U.S. Application Data
(60) Provisional application No. 62/971,088, filed on Feb. 6, 2020.

(51) Int. Cl. A61M 11/02; A61M 16/06

(52) U.S. Cl. CPC A61M 11/02; A61M 16/06; A61M 2202/02

(57) ABSTRACT

Drug delivery hubs are described herein. The drug delivery hub includes a hub housing, a reservoir, a nebulizer channel, and a gas channel. The hub housing defines a housing volume. The hub housing is configured to directly couple with a mask defining a patient cavity. The reservoir is disposed at least partially within the housing volume. The reservoir includes a reservoir volume configured to dispense a medicament. The nebulizer channel is in fluid communication with the reservoir volume and configured to be in fluid communication with the patient cavity when the hub is coupled with the mask. The gas channel is in fluid communication with the nebulizer channel. The gas channel is configured to direct a gas flow toward the patient cavity through the nebulizer channel, drawing a portion of the medicament from the reservoir through the nebulizer channel with the gas flow toward the patient cavity.

### CROSS-REFERENCE TO RELATED APPLICATIONS

This application is the national stage application pursuant to 35 U. S. C. 371 of International Patent Application No. PCT/US2021/015868, entitled “DRUG DELIVERY HUB FOR VENTILATION MASK,” filed on Jan. 29, 2021, which claims priority to U.S. Provisional Application No. 62/971,088, entitled “DRUG DELIVERY HUB FOR VENTILATION MASK,” filed on Feb. 6, 2020, the disclosure of which is incorporated herein by reference in its entirety.

### FIELD OF THE INVENTION

The present disclosure generally relates to nebulizer devices, and, in particular, to nebulizer devices configured to couple with a ventilation mask.

### BACKGROUND

Supplemental gas (e.g., air or oxygen) delivery to patients is a well-known treatment for a number of illnesses and conditions. For patients with respiratory difficulties, oxygen may be provided from an oxygen supply (e.g., pressurized cylinder, etc.) through a ventilation mask. During respiratory therapy or support with a ventilation mask, it is often desirable to also treat a patient with therapeutic drugs or medicaments.

In some applications, during supplemental gas therapy administered via a ventilation mask, administration of a therapeutic medicament can interrupt or diminish the effectiveness of the supplemental gas therapy. Interrupting the supplemental gas therapy to administer a medicament can also diminish the effectiveness of the medicament itself.

### SUMMARY

The disclosed subject matter relates to drug delivery hubs. In certain embodiments, a drug delivery hub is disclosed that comprises a hub housing defining a housing volume, wherein the hub housing is configured to directly couple with a mask defining a patient cavity; a reservoir disposed at least partially within the housing volume, wherein the reservoir comprises a reservoir volume configured to dispense a medicament; a nebulizer channel in fluid communication with the reservoir volume and configured to be in fluid communication with the patient cavity when the hub is coupled with the mask; and a gas channel in fluid communication with the nebulizer channel, wherein the gas channel is configured to direct a gas flow toward the patient cavity through the nebulizer channel, drawing a portion of the medicament from the reservoir through the nebulizer channel with the gas flow toward the patient cavity.

In certain embodiments, a ventilation mask is disclosed that comprises a mask body defining a patient cavity, the mask body comprising a patient opening in fluid communication with the patient cavity; and a drug delivery hub, comprising: a hub housing defining a housing volume, wherein the hub housing is directly coupled to the mask body; a reservoir disposed at least partially within the housing volume, wherein the reservoir comprises a reservoir volume configured to dispense a medicament; a nebulizer channel in fluid communication with the reservoir volume and configured to be in fluid communication with the patient cavity; and a gas channel in fluid communication with the nebulizer channel, wherein the gas channel is configured to direct a gas flow toward the patient cavity through the nebulizer channel, drawing a portion of the medicament from the reservoir through the nebulizer channel with the gas flow toward the patient cavity.

In certain embodiments, a ventilation mask is disclosed that comprises a mask body defining a patient cavity, the mask body comprising a patient opening in fluid communication with the patient cavity; a therapeutic gas manifold coupled to the mask body, the therapeutic gas manifold defining a therapeutic gas channel, the therapeutic gas manifold comprising a plurality of therapeutic gas ports in fluid communication with the therapeutic gas channel, wherein the plurality of therapeutic gas ports are configured to create a therapeutic gas flow within the patient cavity; and a drug delivery hub, comprising: a hub housing defining a housing volume, wherein the hub housing is disposed between the mask body and the therapeutic gas manifold; a reservoir disposed at least partially within the housing volume, wherein the reservoir comprises a reservoir volume configured to dispense a medicament; a nebulizer channel in fluid communication with the reservoir volume and configured to be in fluid communication with the patient cavity; and a gas channel in fluid communication with the nebulizer channel, wherein the gas channel is configured to direct a gas flow toward the patient cavity through the nebulizer channel, drawing a portion of the medicament from the reservoir through the nebulizer channel with the gas flow toward the patient cavity.

In certain embodiments, a method is disclosed that comprises introducing a therapeutic gas into a patient cavity of a ventilation mask via a plurality of gas ports; directing a gas flow through a nebulizer channel; drawing a portion of a medicament from a reservoir through the nebulizer channel with the gas flow, wherein the reservoir is disposed within a drug delivery hub coupled to the ventilation mask; and directing the gas flow and the portion of the medicament into the patient cavity.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is an exploded perspective view of a ventilation mask with a drug delivery hub, in accordance with various aspects of the present disclosure.

FIG. 2 is a front elevation view of a drug delivery hub for use with the ventilation mask of FIG. 1, in accordance with various aspects of the present disclosure.

FIG. 3 is a cross-sectional view of the drug delivery hub of FIG. 2, in accordance with various aspects of the present disclosure.

FIG. 4 is an exploded perspective view of a ventilation mask with a drug delivery hub, in accordance with various aspects of the present disclosure.

FIG. 5 is an exploded perspective view of a ventilation mask with a drug delivery hub, in accordance with various aspects of the present disclosure.

FIG. 6 is an exploded perspective view of a ventilation mask with a drug delivery hub, in accordance with various aspects of the present disclosure.

FIG. 7 is an exploded side elevation view of a ventilation mask with a cross-sectional view of a drug delivery hub, in accordance with various aspects of the present disclosure.

FIG. 8 is a front elevation view of a drug delivery hub for use with the ventilation mask of FIG. 7, in accordance with various aspects of the present disclosure.

FIG. 9 is a cross-sectional view of the drug delivery hub of FIG. 8, in accordance with various aspects of the present disclosure.

FIG. 10 is a perspective view of a ventilation mask, in accordance with various aspects of the present disclosure.

### DETAILED DESCRIPTION

The disclosed drug delivery hub incorporates features to permit the administration of a medicament without interrupting the supplemental gas therapy or diminishing the effectiveness of the supplemental gas therapy and/or the administered drugs. The drug delivery hub can be coupled to a ventilation mask and can dispense a medicament within a flow path defined within the hub housing to generate a medicament aerosol in the immediate vicinity of the patient's airway.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to the administration of supplemental gas and/or a medicament to a patient using the disclosed drug delivery hub, it is to be understood that this description is only an example of usage and does not limit the scope of the claims. Various aspects of the disclosed drug delivery hub may be used in any application where it is desirable to administer a supplemental gas and/or a medicament.

The disclosed drug delivery hub overcomes several challenges discovered with respect to certain medication delivery devices. One challenge with certain conventional medication delivery devices, such as nebulizers, metered-dose inhalers (MDIs), and dry-powder inhalers is that such devices are primarily configured for handheld use and may obstruct or otherwise prevent the use of ventilation masks. As a result, ventilation masks are often removed during medication delivery, which may disturb the respiratory therapy and complicate caregiver workflow. In some applications, certain conventional nebulization masks can receive aerosol from a jet nebulizer via a receiving port. One challenge with certain conventional nebulization masks is that the output of the nebulizer can frequently be too locationally concentrated in a flow field that may not be appropriately directed for patient breathing (e.g. flow may be directed toward a patient's eyes), sacrificing delivery efficiency. Another challenge with certain conventional nebulization masks is that flow requirements of the nebulizer may differ from the patient's flow requirements. Further, certain conventional nebulization masks may not provide sufficient oxygen delivery during non-nebulizing periods.

Therefore, in accordance with the present disclosure, it is advantageous to provide a drug delivery hub as described herein that allows for the administration of medicament during supplemental gas (e.g. oxygen) therapy. In some embodiments, the drug delivery hub can engage with or couple with a ventilation mask, providing hands-free drug delivery. In some embodiments, the drug delivery hub can access pneumatic driving gas for nebulization though docking or sharing of gas from a ventilation mask. In some embodiments, the drug delivery hub provides distributed flow of nebulized drugs to the patient's mouth and/or nose. Further, in some embodiments, the drug delivery hub can provide simultaneous delivery of nebulized drugs and supplemental gas without the need for any additional adapters.

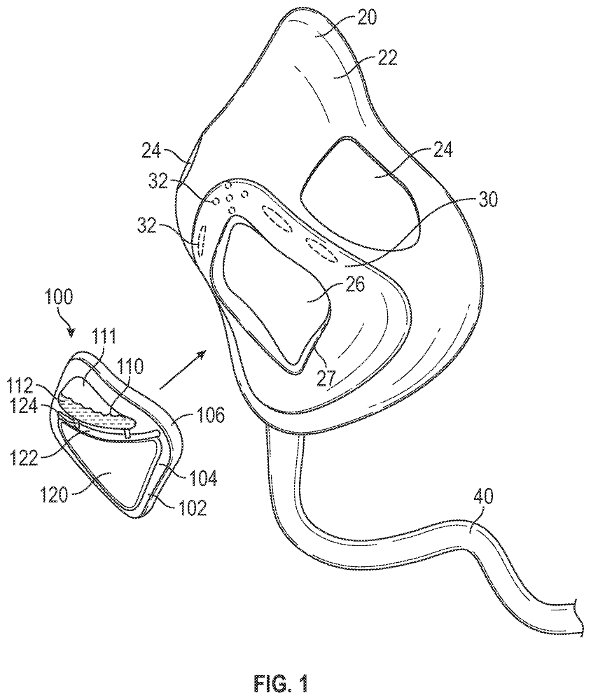

Examples of drug delivery hubs that allow for the administration of medicament during supplemental gas therapy are now described. FIG. 1 is an exploded perspective view of a ventilation mask 20 with a drug delivery hub 100, in accordance with various aspects of the present disclosure. In the depicted example, the drug delivery hub 100 can provide medicament aerosol to a patient without interrupting therapeutic gas flow from the ventilation mask 20 or otherwise removing the ventilation mask 20.

In the depicted example, the ventilation mask 20 can be utilized to administer oxygen or other supplemental gases to a patient. As illustrated, the ventilation mask 20 can be worn by the patient, defining a patient cavity over the patient's mouth and nose. The ventilation mask 20 can define an inner surface within the patient cavity and an outer surface opposite to the inner surface. The ventilation mask 20 can direct a supplemental gas, such as oxygen, via the supply line 40 to the ventilation mask 20.

In some embodiments, a gas manifold 30 can distribute the supplemental gas through the mask body 22 to the patient. In the depicted example, supplemental gases can be introduced into the patient cavity via one or more gas ports 32, formed in the gas manifold 30 disposed within the patient cavity of the mask body 22. The gas ports 32 can be disposed along the inner surface of the ventilation mask 20. In some applications, the gas ports 32 can administer high concentrations of supplemental gas to the patient cavity and ultimately to the patient.

In the depicted example, the ventilation mask 20 can have a generally open mask structure. As illustrated, the mask body 22 includes one or more vent openings 24, 26 formed therethrough. The vent openings 24, 26 can allow for access or fluid communication with the patient cavity defined by the mask body 22. In some embodiments, the mask body 22 includes three vent openings 24, 26. The upper vent openings 24 can be positioned to be adjacent to a patient's nose when the ventilation mask 20 is worn. Further, the upper vent openings 24 can be laterally spaced apart on either side of the patient's nose when the ventilation mask 20 is worn. The lower vent opening 26 can be positioned to be adjacent to a patient's mouth when the ventilation mask 20 is worn. In some embodiments, the ventilation mask 20 includes one or more of the vent openings 24, 26.

Advantageously, by utilizing one or more vent openings 24, 26, the ventilation mask 20 can allow for exhaled gases such as carbon dioxide to exit from the patient cavity of the ventilation mask, reducing the incidence of carbon dioxide rebreathing by the patient. Further, the vent openings 24, 26 can permit various tasks to be performed without removing the ventilation mask 20. Tasks can include, but are not limited to, medical procedures, eating, drinking, hygiene procedures, and/or talking. For example, the vent openings 24, 26 can allow for nasal and/or oral bronchoscopy procedures, administering medications, and/or access for drug delivery hubs as described herein. Further, the open structure of the ventilation mask 20 can increase patient comfort by accommodating various facial features and reducing patient claustrophobia.

In the depicted example, a drug delivery hub 100 can be coupled or attached to the ventilation mask 20 to deliver or administer a medicament aerosol to the patient without interrupting gas flow from the ventilation mask 20 or removing the ventilation mask 20. Medicaments can include, but are not limited to, surfactants, drugs (e.g. antibiotics, anti-viral, etc.), polymers, naturopathic remedies, and/or homeopathic remedies. The drug delivery hub 100 can be coupled or attached to the outer surface of the ventilation mask 20. Optionally, the drug delivery hub 100 can be coupled or attached to the inner surface of the ventilation mask 20. The drug delivery hub 100 can include a hub housing 102, defining a housing volume 104 therein. In some embodiments, components of the drug delivery hub 100, such as the reservoir 110, the gas channel 120, the nebulization portion 122, and the medicament channel 124 can be contained within the housing volume 104.

In some embodiments, the drug delivery hub 100 can be removably attached to the ventilation mask 20 to provide medicament aerosol when attached to the ventilation mask 20, and to allow for normal operation of the ventilation mask 20 when the drug delivery hub 100 is separated from the ventilation mask 20. In the depicted example, the drug delivery hub 100 can be attached to a vent opening 24, 26 of the ventilation mask 20. As illustrated, the drug delivery hub 100 can be coupled to the lower vent opening 26 of the ventilation mask 20. In some embodiments, the drug delivery hub 100 can have a hub housing 102 defining a continuous surface. In some applications, the hub housing 102 may at least partially obstruct the respective vent opening 24, 26 to which the drug delivery hub 100 is coupled. In some embodiments, the shape or structure of the hub housing 102 can allow for a flow to pass through the vent openings 24, 26.

Optionally, the hub perimeter 106 of the hub housing 102 can engage with the opening perimeter 27 of the vent opening 26. The hub perimeter 106 and the opening perimeter 27 can be in sealing engagement to prevent leakage of medicament aerosol and/or supplemental gas flow. In some embodiments, the hub housing 102 can deform to engage with the vent opening 26. In some embodiments, the ventilation mask 20 can deform to engage with the drug delivery hub 100. As can be appreciated, both the drug delivery hub 100 and the ventilation mask 20 can deform to allow the drug delivery hub 100 and the ventilation mask 20 to engage together and/or disengage. In some embodiments, the drug delivery hub 100 can resiliently engage or “snap in” with the ventilation mask 20.

As described herein, the drug delivery hub 100 provides nebulizing or aerosolized medicament 112 to the patient. In the depicted example, the drug delivery hub 100 receives gas flow (e.g., air, oxygen, and/or another gas or combination thereof) from a gas source. As described herein, the gas source can be an independent gas source or a gas source that is shared with the ventilation mask 20. In some embodiments, the drug delivery hub 100 can receive a gas flow from the supply line 40.

In the depicted example, gas flow can move into the gas channel 120. As can be appreciated, the gas channel 120 can be defined or formed within the housing volume 104 of the hub housing 102. In the depicted example, gas flow received by the gas channel 120 can be utilized by the drug delivery hub 100 to generate medicament 112 aerosol or nebulizer flow within the housing volume 104 to be directed to the patient cavity defined by the ventilation mask 20 or otherwise administered to the patient. As described herein, the drug delivery hub 100 can be a pneumatic aerosol generator that utilizes accelerated flow to draw medicament 112 out of a reservoir volume 111 defined by the reservoir 110 and atomizes the medicament 112 into an aerosol. Advantageously, by utilizing an aerosol generator within the housing volume 104, the medicament aerosol or nebulizer flow is generated in the immediate vicinity of the point of gas flow entry into the patient's airway. It is also contemplated that any embodiment of the present disclosure can be used with a dry powder medicament such that the accelerated gas flow causes the medicament to be pulverized and/or incorporated into the gas flow and directed toward the patient.

As can be appreciated, in some embodiments, the gas flow through the drug delivery hub 100 may be controlled to provide for increased efficiency and/or consistent droplet size production. For example, in some embodiments, the gas flow may be limited to a range between approximately 2 L/min to approximately 10 L/min to provide for increased efficiency and/or consistent droplet size production. In some embodiments, the gas flow can range between approximately 1 L/min to 20 L/min, approximately 1 L/min to 15 L/min, approximately 1 L/min to 12 L/min, approximately 1 L/min to 10 L/min, approximately 2 L/min to 20 L/min, approximately 2 L/min to 15 L/min, approximately 2 L/min to 12 L/min, approximately 2 L/min to 8 L/min, and approximately 2 L/min to 5 L/min.

In the illustrated embodiment, medicament 112 is drawn from the reservoir 110. As described herein, as gas flow moves from the gas channel 120 into the nebulization portion 122, the velocity of the gas flow within the nebulization portion 122 can increase, decreasing the fluid pressure of the gas flow within the nebulization channel (e.g., by creating a Venturi effect). As can be appreciated, the decreased fluid pressure of the gas flow within the nebulization portion 122 can draw medicament 112 into the nebulization portion 122 from the reservoir 110 via the medicament channel 124.

As illustrated, the reservoir 110 can be at least partially disposed within the housing volume 104. In some embodiments, the reservoir 110 is disposed at or near a top portion of the drug delivery hub 100. Optionally, the reservoir 110 can be disposed in any portion of the drug delivery hub 100, including the bottom portion or the lateral portions of the drug delivery hub 100.

Optionally, the reservoir 110 can be removable or removably attached to the drug delivery hub 100. The reservoir volume 111 can contain a liquid or powdered medicament 112.

After drawing the medicament 112 from the reservoir 110 into the nebulization portion 122, the force of the gas flow can accelerate and shear the medicament 112, forming droplets of medicament 112 which combine with the gas flow to form the nebulizing gas flow. As described herein, the flow rate of the gas flow can be controlled to permit the desired formation of medicament 112 droplets or aerosol appropriate for delivery into the patient's airways and/or lungs. As described here, the nebulizing gas flow can be directed into the patient cavity of the ventilation mask 20 to be inhaled by the patient's mouth and/or nose.

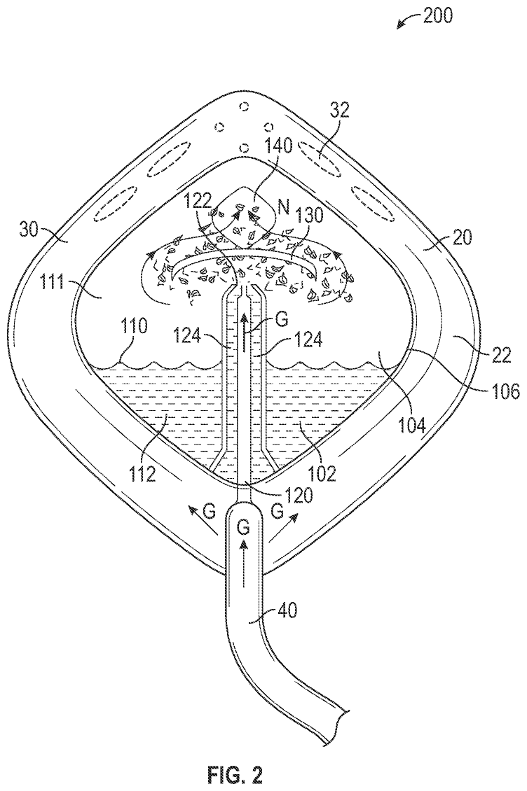

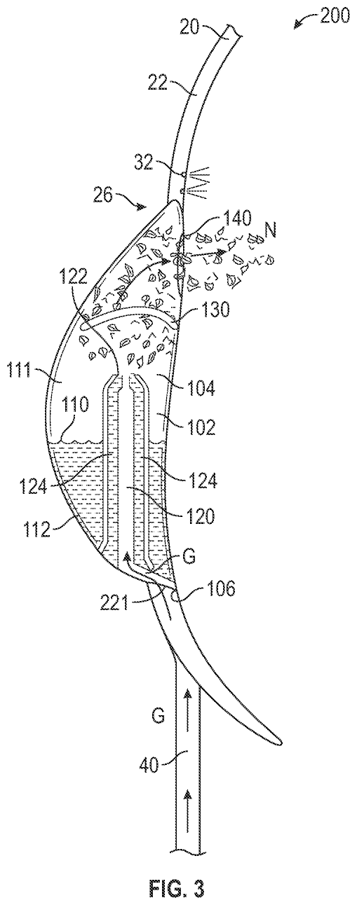

FIG. 2 is a front elevation view of a drug delivery hub 200 for use with the ventilation mask 20 of FIG. 1, in accordance with various aspects of the present disclosure. FIG. 3 is a cross-sectional view of the drug delivery hub 200 of FIG. 2, in accordance with various aspects of the present disclosure. The drug delivery hub 200 can include features that are similar to drug delivery hub 100 and can operate in a similar manner. Unless noted, similar features of drug delivery hub 200 can be referred to with the same reference numerals as discussed with reference to drug delivery hub 100. With reference to FIGS. 2 and 3, the drug delivery hub 200 facilitates the nebulization of medicament 112 by drawing the medicament from a reservoir 110 toward a nebulization portion 122 through capillary action.

In the depicted example, medicament 112 is stored in a reservoir 110 defined within the housing volume 104. As illustrated, the reservoir 110 can utilize the housing volume 104 as the reservoir volume 111, storing medicament 112 in a lower portion of the housing volume 104. In some embodiments, the reservoir volume 111 is pre-filled with medicament 112. Optionally, medicament 112 can be introduced or metered into the reservoir 110 through a medicament delivery line. In some embodiments, the reservoir 110 can include an access port to allow manual administration of medicament 112 into the reservoir 110 to fill and/or refill with reservoir 110. Further, the access port can allow for the introduction of an ampule or a cartridge containing a medicament. In some embodiments, the reservoir 110 can be disposed at least partially or completely within the housing volume 104.

In the depicted example, gas flow G is introduced into the drug delivery hub 200 from the gas supply line 40. As illustrated, the gas flow G can be introduced into the drug delivery hub 200 and/or the gas channel 120 at a bottom portion of the drug delivery hub 200. In some embodiments, the gas supply line 40 is attached to the drug delivery hub 200 by an access port 221. The access port 221 can include a self-sealing membrane and/or a luer connection to seal the access port 221 when the drug delivery hub 200 is not connected to the gas supply line 40. During operation, the self-sealing membrane or luer connection can allow fluid communication between the gas supply line 40 and the gas channel 120. Optionally, the gas flow G from the gas supply line 40 can be shared between the drug delivery hub 200 and the gas manifold 30.

In the depicted example, the gas flow G can be directed upward through the gas channel 120 toward an upper portion of the drug delivery hub 200. In some embodiments, the gas channel 120 is centrally located within the housing volume 104. During operation, the drug delivery hub 200 directs gas flow G from the gas channel 120 toward the nebulizing portion 122. As illustrated, the nebulizing portion 122 of the gas channel 120 can have a constricting geometry or otherwise reduced diameter relative to the gas channel 120 to increase the velocity of the gas flow G. As can be appreciated, as the velocity of the gas flow G is increased within the nebulizing portion 122, the fluid pressure of the gas flow G within the nebulizing portion 122 is reduced (e.g., a Venturi effect). As can be appreciated, the decreased fluid pressure of the gas flow within the nebulization portion 122 can draw medicament 112 from the medicament channel 124 into the nebulizing portion 122.

In some embodiments, the medicament 112 can be drawn upward from the reservoir 110 toward the nebulizing portion 122 through a medicament channel 124. The medicament channel 124 can utilize capillary action to draw the medicament 112 upward. Optionally, the medicament channel 124 can have a generally annular shape formed around the gas channel 120. In some embodiments, the medicament channel 124 can extend into the lower or base portion of the reservoir 110, allowing the medicament channel 124 to draw medicament 112 from the reservoir 110 as the medicament 112 is consumed or nebulized.

As the medicament 112 is drawn into the nebulizing portion 122 by the gas flow G, the force of the high-speed gas flow G can accelerate and shear the medicament 112, forming droplets of medicament 112 which combine with the gas flow G to form the nebulizing gas flow N. As described herein, the flow rate of the gas flow G can be controlled to permit the desired formation of medicament 112 droplets or aerosol appropriate for delivery into the patient's airways and/or lungs.

In some embodiments of the present disclosure, the flow rate of the gas flow G can be affected by the cross-sectional width of the gas channel 120. In some embodiments, the gas channel 120 can include a constriction or narrower portion relative to the gas supply line 40. In some embodiments, a removable constriction can be positioned in the gas channel 120 to achieve a specific gas flow G rate. The removable constriction (not shown) can be a baffle, an orifice or an o-ring positioned in the gas channel 120 at the access port 221.

In the depicted example, the drug delivery hub 200 includes a baffle 130 to promote disintegration of the liquid medicament 112 within the nebulizing gas flow N. As illustrated, the baffle 130 can be disposed within the housing volume 104 such that nebulizing gas flow N impinges upon the baffle 130. The baffle 130 can be positioned between the nebulizing portion 122 and the flow outlet 140, preventing direct flow of nebulizing gas flow N into the flow outlet 140. Further, the baffle 130 can allow larger droplets to be captured and redirected toward the reservoir 110.

In some embodiments, at least one baffle 130 can be disposed adjacent to the exit of the nebulizing portion 122. Optionally, the baffle 130 can be spaced apart from the exit of the nebulizing portion 122. In some embodiments, the baffle 130 can generally have a shield or umbrella shape. As can be appreciated, the baffle 130 can be formed as any suitable shape. As can be appreciated, the positioning and the geometry of the baffle 130 can be adjusted relative to the nebulizing portion 122 to promote droplet formation and additional pulverization within the nebulizing gas flow N.

During operation, the nebulizing gas flow N can flow into the patient cavity defined by the ventilation mask 20 through the flow outlet 140. As can be appreciated, the nebulizing gas flow N can mix with the supplemental gas flow provided by the gas ports 32 to be inhaled by the patient.

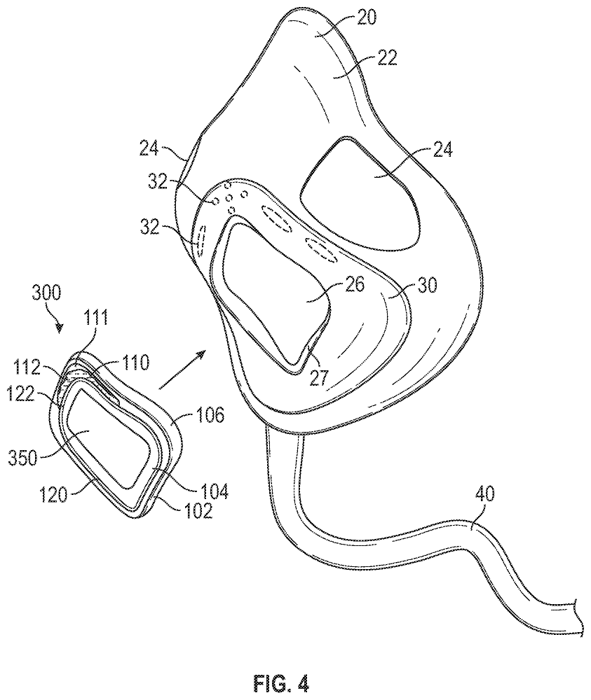

FIG. 4 is an exploded perspective view of a ventilation mask 20 with a drug delivery hub 300, in accordance with various aspects of the present disclosure. The drug delivery hub 400 can include features that are similar to drug delivery hub 100 and can operate in a similar manner. Unless noted, similar features of drug delivery hub 400 can be referred to with the same reference numerals as discussed with reference to drug delivery hub 100. In the depicted example, the drug delivery hub 300 can have an opening 350 to allow the ventilation mask 20 to permit access into the patient cavity through the lower vent opening 26. Advantageously, the opening 350 can allow the patient to continue to speak, drink, or perform other activities during nebulization procedures.

As illustrated, the drug delivery hub 300 can have a generally ring-like structure forming the opening 350 therethrough. The opening 350 can be defined by an inner surface of the drug delivery hub 300. The drug delivery hub 300 can have a continuous perimeter defining the opening 350 therein, or can have a semi-continuous perimeter, such as a U-shape, defining the opening 350 therein. In the depicted example, the components of the drug delivery hub 300 are disposed around the perimeter of the opening 350, permitting the formation of the opening 350 and the nebulizing functionality described herein. In some embodiments, the gas channel 120 can be formed generally around the opening 350. Optionally, the reservoir 110 can be disposed around the opening 350 or away from the opening 350.

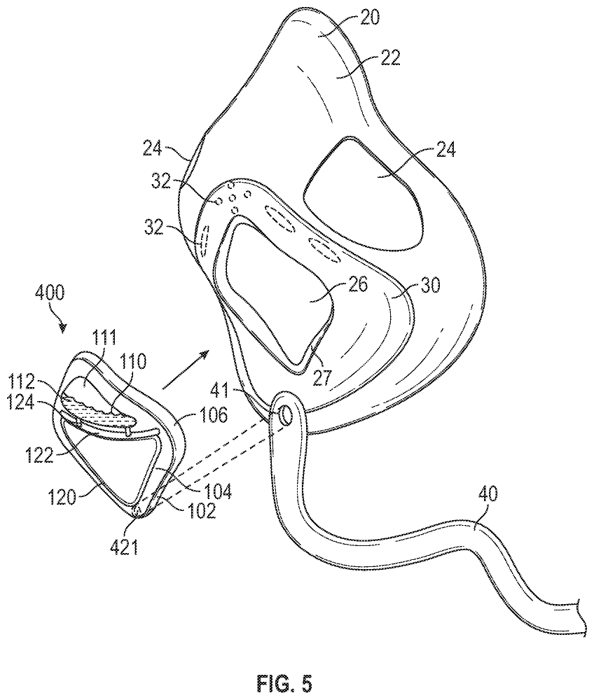

FIG. 5 is an exploded perspective view of a ventilation mask 20 with a drug delivery hub 400, in accordance with various aspects of the present disclosure. The drug delivery hub 400 can include features that are similar to drug delivery hub 100 and can operate in a similar manner. Unless noted, similar features of drug delivery hub 400 can be referred to with the same reference numerals as discussed with reference to drug delivery hub 100. In the depicted example, the gas channel 120 of the drug delivery hub 400 receives gas flow from the supply line 40. As illustrated, the drug delivery hub 400 can include a port 421 to receive gas flow from a port 41 formed in the supply line 40. As can be appreciated, the port 421 can be aligned to engage with the port 41 when the drug delivery hub 400 is engaged or otherwise coupled with the ventilation mask 20. Optionally, the port 421 can be formed as a part of the hub housing 102.

In some embodiments, gas within the supply line 40 can be shared between the drug delivery hub 400 and the ventilation mask 20. As can be appreciated, a portion of the gas flow can exit the supply line 40 at the port 41 and another portion of gas flow can continue into the ventilation mask 20. In some embodiments, the port 41 and/or the port 421 can include a self-sealing membrane or a luer connection to allow for the port 41 and/or the port 421 to seal when not in use.

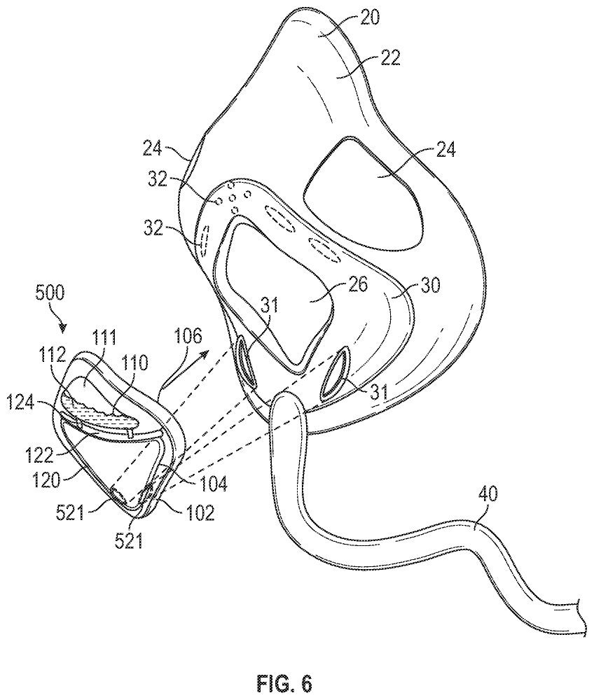

FIG. 6 is an exploded perspective view of a ventilation mask 20 with a drug delivery hub 500, in accordance with various aspects of the present disclosure. In the depicted example, the gas channel 120 of the drug delivery hub 500 receives gas flow from the ports 31 formed on the ventilation mask 20. The drug delivery hub 500 can include features that are similar to drug delivery hub 100 and can operate in a similar manner. Unless noted, similar features of drug delivery hub 500 can be referred to with the same reference numerals as discussed with reference to drug delivery hub 100. As illustrated, the drug delivery hub 500 can include ports 521 to receive gas flow from the ventilation mask 20. As can be appreciated, the ports 521 can be aligned to engage with the ports 31 when the drug delivery hub 500 is engaged or otherwise coupled with the ventilation mask 20. Optionally, the ports 521 can be formed as a part of the hub housing 102.

In some embodiments, gas from the ventilation mask 20 and/or the gas manifold 30 can be shared with the drug delivery hub 500. As can be appreciated, gas can flow into the ventilation mask 20 from the supply line 40 to deliver supplemental gas flow to the patient via the gas ports 32 and deliver gas flow to the drug delivery hub 500 via the ports 31. In some embodiments, the ports 31 and/or the ports 521 can include a self-sealing membrane or a luer connection to allow for the port 31 and/or the port 521 to seal when not in use.

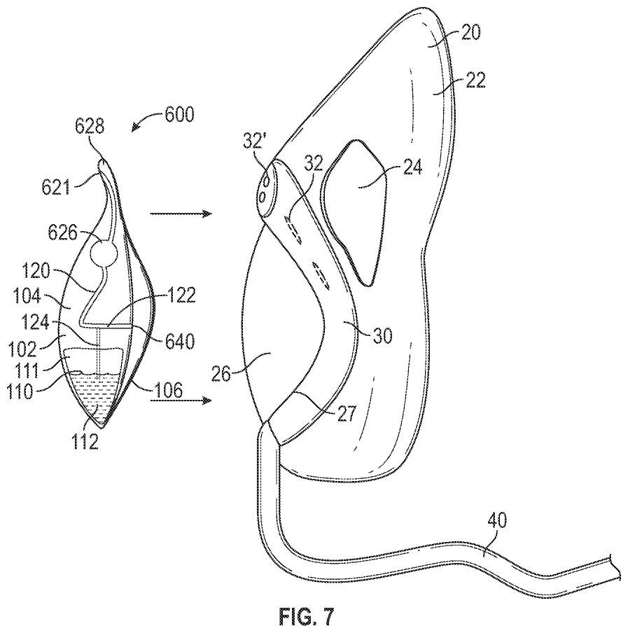

FIG. 7 is an exploded side elevation view of a ventilation mask 20 with a cross-sectional view of a drug delivery hub 600, in accordance with various aspects of the present disclosure. The drug delivery hub 600 can include features that are similar to drug delivery hub 100 and can operate in a similar manner. Unless noted, similar features of drug delivery hub 600 can be referred to with the same reference numerals as discussed with reference to drug delivery hub 100. In the depicted example, the drug delivery hub 600 can receive gas flow for nebulization from the gas ports 32′ of the ventilation mask 20 that are typically used for delivering supplemental gas flow to the patient. As illustrated, when the drug delivery hub 600 is coupled to the ventilation mask 20, the drug delivery hub 600 is shaped or otherwise configured to allow the gas channel 120 to be in fluid communication with one or more of the gas ports 32′ of the ventilation mask 20. Optionally, the gas channel 120 can include a flow feature 626 to adjust the flow within the gas channel 120. As illustrated, nebulizing flow can enter the patient cavity via the outlet 640.

In some embodiments, the reservoir 110 can be disposed toward a lower portion of the hub housing 102. In some embodiments, the reservoir 110 can be disposed below the gas channel 120, the medicament channel 124, and the nebulization portion 122. Advantageously, by locating the reservoir 110 below other components of the drug delivery hub 600, the hub housing 102 may be configured to be more compact.

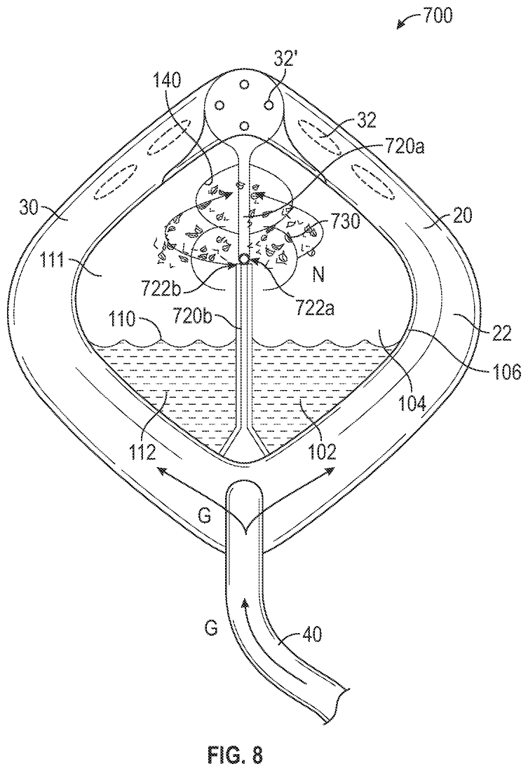

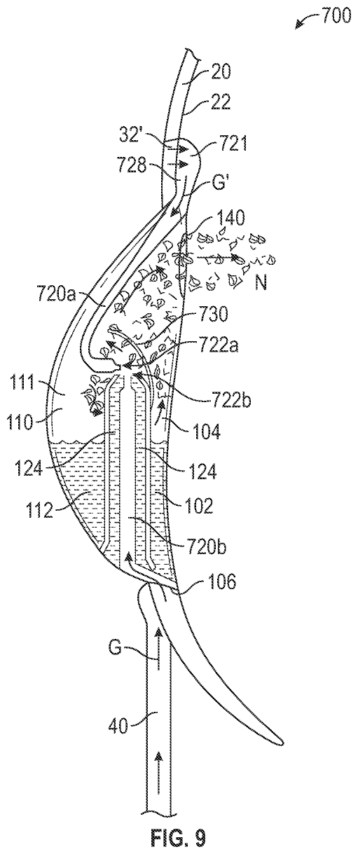

FIG. 8 is a front elevation view of a drug delivery hub 700 for use with the ventilation mask 20 of FIG. 7, in accordance with various aspects of the present disclosure. FIG. 9 is a cross-sectional view of the drug delivery hub 700 of FIG. 8, in accordance with various aspects of the present disclosure. The drug delivery hub 700 can include features that are similar to drug delivery hub 600 and can operate in a similar manner. Unless noted, similar features of drug delivery hub 700 can be referred to with the same reference numerals as discussed with reference to drug delivery hub 600. With reference to FIGS. 8 and 9, the drug delivery hub 700 facilitates nebulization by drawing gas flow from gas ports 32′ of the ventilation mask 20. Similar to drug delivery hub 200, the medicament 112 can be stored in a reservoir 110 defined within the housing volume 104.

In the depicted example, gas flow G′ is introduced into the drug delivery hub 700 from the gas ports 32′ of the ventilation mask 20. In the depicted example, the access port extension 728 of the drug delivery hub 700 extends into an inner portion of the ventilation mask 20 to align or otherwise engage the access ports 721 of the access port extension 728 with one or more of the gas ports 32′ of the ventilation mask 20. Optionally, the access port extension 728 can seal against an inner surface of the ventilation mask 20 adjacent to the gas ports 32′. The access ports 721 can include a self-sealing membrane. Advantageously, the access ports 721 can capture flow from a portion of the gas ports 32′, allowing for flow to be split between the drug delivery hub 700 and gas flow into the patient cavity.

In the depicted example, the gas flow G′ can be directed downward through a gas channel 720a toward a lower portion of the drug delivery hub 700. During operation, the gas channel 720a can direct gas flow G′ toward the nebulizing portion 722a. As illustrated, the nebulizing portion 722a of the gas channel 720a can have a constricting geometry or otherwise reduced diameter relative to the gas channel 720a to increase the velocity of the gas flow G′. As can be appreciated, as the velocity of the gas flow G′ is increased within the nebulizing portion 722a, the fluid pressure of the gas flow G′ within the nebulizing portion 722a is reduced (e.g., a Venturi effect). As can be appreciated, the decreased fluid pressure of the gas flow within the nebulization portion 722a can draw medicament 112 from the medicament channel 124 toward the nebulizing portion 722a. In some embodiments, the gas channel 720a can be directed toward the patient or away from the patient.

Additionally or alternatively, the gas flow G can also be introduced into the drug delivery hub 700 by a gas channel 720b at a bottom portion of the drug delivery hub 700. In some embodiments, the gas supply line 40 is attached to the drug delivery hub 700 by an access port. In some embodiments, the gas flow G from the gas supply line 40 can be shared between the drug delivery hub 700 and the gas manifold 30. It should be understood that the drug delivery hub of the present disclosure can be configured with either a gas channel 720a to direct a gas G′ toward a nebulizing portion 722a, or a gas channel 720b to direct a gas G toward a nebulizing portion 722b. It should also be understood that the drug delivery hub of the present disclosure can be configured with a gas channel 720a and a gas channel 720b to direct a gas to a nebulizing portions 722a, 722b.

In the depicted example, the gas flow G can be directed upward through the gas channel 720b toward an upper portion of the drug delivery hub 700. During operation, the drug delivery hub 700 directs gas flow G from the gas channel 720b toward the nebulizing portion 722b. As illustrated, the nebulizing portion 722b of the gas channel 720b can have a constricting geometry or otherwise reduced diameter relative to the gas channel 720b to increase the velocity of the gas flow G. As can be appreciated, as the velocity of the gas flow G is increased within the nebulizing portion 722b, the fluid pressure of the gas flow G within the nebulizing portion 722b is reduced (e.g., a Venturi effect). In addition to the gas flow G from the gas channel 720a the decreased fluid pressure of the gas flow within the nebulization portion 722b can draw medicament 112 from the medicament channel 124 into the nebulizing portion 722b.

In some embodiments, the medicament 112 can be drawn upward from the reservoir 110 toward the nebulizing portion 722a, 722b through a medicament channel 124. The medicament channel 124 can utilize capillary action to draw the medicament 112 upward. Optionally, the medicament channel 124 can have a generally annular shape formed around the gas channel 720b. In some embodiments, the medicament channel 124 can extend into the lower or base portion of the reservoir 110, allowing the medicament channel 124 to draw medicament 112 from the reservoir 110 as the medicament 112 is consumed or nebulized.

As the medicament 112 is drawn toward the nebulizing portion 722a, 722b by the gas flow G, the force of the high-speed gas flow G can accelerate and shear the medicament 112, forming droplets of medicament 112 that combine with the gas flow G to form the nebulizing gas flow N. As described herein, the flow rate of the gas flow G can be controlled to permit the desired formation of medicament 112 droplets or aerosol appropriate for delivery into the patient's airways and/or lungs.

In the depicted example, the drug delivery hub 700 includes a baffle 730 to promote atomization of the liquid medicament 112, or pulverization of a dry medicament, within the nebulizing gas flow N. As illustrated, the baffle 730 can be disposed within the housing volume 104 such that nebulizing gas flow N impinges upon the baffle 730. The baffle 730 can be positioned between the nebulizing portion 722a, 722b and the flow outlet 140, preventing direct flow of nebulizing gas flow N into the flow outlet 140. Further, the baffle 730 can allow larger droplets to be captured and redirected toward the reservoir 110. In some embodiments, at least one baffle 730 can be disposed adjacent to the exit of the nebulizing portion 722a, 722b. Optionally, the baffle 730 can be spaced apart from the exit of the nebulizing portion 722a, 722b. In some embodiments, the baffle 730 can generally have a shield or umbrella shape. As can be appreciated, the baffle 730 can be formed as any suitable shape. As can be appreciated, the positioning and the geometry of the baffle 730 can be adjusted relative to the nebulizing portion 722a, 722b to promote droplet formation and additional pulverization within the nebulizing gas flow N. Optionally, the walls of the hub housing 102 can be shaped to function as a baffle.

During operation, the nebulizing gas flow N can flow into the patient cavity defined by the ventilation mask 20 through the flow outlet 140. As can be appreciated, the nebulizing gas flow N can mix with the supplemental gas flow provided by the gas ports 32 to be inhaled by the patient.

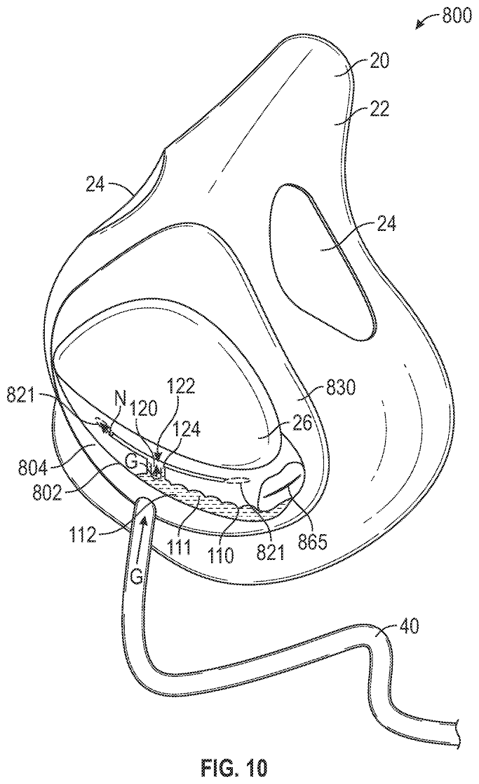

FIG. 10 is a perspective view of a ventilation mask 800, in accordance with various aspects of the present disclosure. The ventilation mask 800 can include features that are similar to drug delivery hub 100 and can operate in a similar manner. Unless noted, similar features of ventilation mask 800 can be referred to with the same reference numerals as discussed with reference to drug delivery hub 100. In the depicted example, the ventilation mask 800 includes an integrated nebulizer to provide supplemental gas flow and/or nebulizing gas flow as required.

In the depicted example, medicament 112 is stored in a reservoir 110 defined within the nebulizer manifold 830. As illustrated, the reservoir 110 can utilize the nebulizer manifold 830 as the reservoir volume 111, storing medicament 112 in a lower portion of the nebulizer manifold 830. In some embodiments, the reservoir volume 111 is pre-filled with medicament 112. In some embodiments, the nebulizer manifold 830 can include a fill port 865 to allow manual administration of medicament 112 into the reservoir 110 to fill and/or refill reservoir 110. Advantageously, the ventilation mask 800 can function as a ventilation mask when the reservoir 110 is empty and provide nebulization functionality when the reservoir 810 is filled with medicament 112.

In the depicted example, gas flow G is introduced into the ventilation mask 800 from the gas supply line 40. As illustrated, the gas flow G can be introduced into the ventilation mask 800 to delivery gas flow G for nebulization and for supplemental gas therapy. Gas flow G can be shared between nebulization and supplemental gas therapy. In some embodiments, the gas flow G, or a portion thereof, is directed into the gas channel 120 and then toward the patient via access ports 821.

During operation, the ventilation mask 800 directs gas flow G through a portion of the gas channel 120 having a constricting geometry or otherwise reduced diameter relative to another portion of the gas channel to increase the velocity of the gas flow G. As can be appreciated, as the velocity of the gas flow G is increased, the fluid pressure of the gas flow G is reduced (e.g., a Venturi effect). The decreased fluid pressure of the gas flow within the gas channel can draw medicament 112 toward the gas channel 120.

In some embodiments, the medicament 112 can be drawn, by gas flow G through the gas channel 120, from the reservoir 110 through a medicament channel 124, similar to the gas channel 720b and the medicament channel 124 described with reference to FIG. 9. In some embodiments, the medicament channel 124 can extend into the lower or base portion of the reservoir 110, allowing the medicament channel 124 to draw medicament 112 from the reservoir 110 as the medicament 112 is consumed or nebulized.

As the medicament 112 is drawn into the nebulizing portion 122 by the gas flow G, the force of the high-speed gas flow G can accelerate and shear the medicament 112, forming droplets of medicament 112 which combine with the gas flow G to form the nebulizing gas flow.

During operation, the nebulizing gas flow N can move into the patient cavity defined by the ventilation mask 800 through the access ports 821. As can be appreciated, the nebulizing gas flow N can mix with the supplemental gas flow provided by the gas ports to be inhaled by the patient.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A drug delivery hub, comprising: a hub housing defining a housing volume, wherein the hub housing is configured to directly couple with a mask defining a patient cavity; a reservoir disposed at least partially within the housing volume, wherein the reservoir comprises a reservoir volume configured to dispense a medicament; a nebulizer channel in fluid communication with the reservoir volume and configured to be in fluid communication with the patient cavity when the hub is coupled with the mask; and a gas channel in fluid communication with the nebulizer channel, wherein the gas channel is configured to direct a gas flow toward the patient cavity through the nebulizer channel, drawing a portion of the medicament from the reservoir through the nebulizer channel with the gas flow toward the patient cavity.

Clause 2. The drug delivery hub of Clause 1, wherein the gas channel receives the gas flow from a gas supply line.

Clause 3. The drug delivery hub of any preceding Clause, wherein the gas channel comprises an access port to receive the gas flow.

Clause 4. The drug delivery hub of Clause 3, wherein the access port is configured to engage with a self-sealing membrane or luer connection.

Clause 5. The drug delivery hub of any preceding Clause, wherein the reservoir is disposed toward an upper portion of the hub housing.

Clause 6. The drug delivery hub of any preceding Clause, wherein the reservoir is disposed toward a lower portion of the hub housing.

Clause 7. The drug delivery hub of any preceding Clause, wherein the reservoir is disposed toward a lateral portion of the hub housing.

Clause 8. The drug delivery hub of any preceding Clause, wherein the reservoir is removably attached to the hub housing.

Clause 9. The drug delivery hub of Clause 8, wherein the reservoir is removably attached to the housing volume.

Clause 10. The drug delivery hub of any preceding Clause, wherein the medicament comprises a liquid medicament.

Clause 11. The drug delivery hub of any preceding Clause, wherein the medicament comprises a powdered medicament.

Clause 12. The drug delivery hub of any preceding Clause, wherein the hub housing is configured to be removably attached to the mask.

Clause 13. The drug delivery hub of any preceding Clause, wherein the hub housing is configured to be sealingly attached to the mask.

Clause 14. The drug delivery hub of any preceding Clause, wherein the hub housing comprises a continuous surface.

Clause 15. The drug delivery hub of any preceding Clause, wherein the hub housing comprises an inner surface that defines a passage through the hub, such that, when the hub is coupled with the mask, the passage extends through the hub to the patient cavity.

Clause 16. The drug delivery hub of any preceding Clause, wherein, when the hub is coupled with a mask, the hub is configured to receive a gas from the mask and direct the gas through the hub to the patient cavity.

Clause 17. The drug delivery hub of any preceding Clause, wherein, when the hub is coupled with a mask, the hub is configured to receive a gas from the mask and direct the gas through the hub and back to the mask.

Clause 18. A ventilation mask, comprising: a mask body defining a patient cavity, the mask body comprising a patient opening in fluid communication with the patient cavity; and
a drug delivery hub, comprising: a hub housing defining a housing volume, wherein the hub housing is directly coupled to the mask body; a reservoir disposed at least partially within the housing volume, wherein the reservoir comprises a reservoir volume configured to dispense a medicament; a nebulizer channel in fluid communication with the reservoir volume and configured to be in fluid communication with the patient cavity; and a gas channel in fluid communication with the nebulizer channel, wherein the gas channel is configured to direct a gas flow toward the patient cavity through the nebulizer channel, drawing a portion of the medicament from the reservoir through the nebulizer channel with the gas flow toward the patient cavity.

Clause 19. The ventilation mask of Clause 18, further comprising: at least one vent opening formed through the mask body, the at least one vent opening in fluid communication with the patient cavity, wherein the at least one vent opening is disposed generally opposite to the patient opening, and the hub housing is coupled to the at least one vent opening.

Clause 20. The ventilation mask of Clause 19, wherein the hub housing comprises a continuous surface and at least partially obstructs the at least one vent opening.

Clause 21. The ventilation mask of Clause 19, wherein the hub housing comprises an inner surface that defines a passage through the hub to the patient cavity.

Clause 22. The ventilation mask of Clause 18-21, wherein the gas channel receives the gas flow from a gas supply line.

Clause 23. The ventilation mask of Clause 18-22, wherein the gas channel comprises an access port to receive the gas flow.

Clause 24. The ventilation mask of Clause 23, wherein the access port is configured to engage with a self-sealing membrane or luer connection.

Clause 25. The ventilation mask of Clause 18-24, further comprising: a therapeutic gas manifold coupled to the mask body, the therapeutic gas manifold defining a therapeutic gas channel, the therapeutic gas manifold comprising a plurality of therapeutic gas ports in fluid communication with the therapeutic gas channel, wherein the plurality of therapeutic gas ports are configured to create a therapeutic gas flow within the patient cavity.

Clause 26. The ventilation mask of Clause 25, wherein the gas channel and the therapeutic gas channel are in fluid communication with a gas supply line.

Clause 27. The ventilation mask of Clause 25, wherein the gas channel receives the gas flow from the therapeutic gas manifold.

Clause 28. The ventilation mask of Clause 27, wherein the gas channel is in fluid communication with at least one of the plurality of therapeutic gas ports.

Clause 29. The ventilation mask of Clause 27, wherein the mask body comprises a therapeutic gas access port formed through the mask body and in fluid communication with the therapeutic gas channel.

Clause 30. The ventilation mask of Clause 29, wherein the gas channel is in fluid communication with the therapeutic gas access port.

Clause 31. The ventilation mask of Clause 25, wherein, when the hub is coupled with a mask, a portion of the hub obstructs at least a portion of the plurality of therapeutic gas ports to scavenge a gas exiting therefrom and direct the scavenged gas into the gas channel of the hub.

Clause 32. The ventilation mask of Clause 29, wherein the therapeutic gas access port comprises a self-sealing membrane.

Clause 33. The ventilation mask of Clause 18, wherein the reservoir is disposed toward an upper portion of the hub housing.

Clause 34. The ventilation mask of Clause 18, wherein the reservoir is disposed toward a lower portion of the hub housing.

Clause 35. The ventilation mask of Clause 18, wherein the reservoir is disposed toward a lateral portion of the hub housing.

Clause 36. The ventilation mask of Clause 18, wherein the reservoir is removably attached to the hub housing.

Clause 37. The ventilation mask of Clause 36, wherein the reservoir is removably attached to the housing volume.

Clause 38. The ventilation mask of Clause 18, wherein the medicament comprises a liquid medicament.

Clause 39. The ventilation mask of Clause 18, wherein the medicament comprises a powdered medicament.

Clause 40. The ventilation mask of Clause 18-39, wherein the hub housing is removably attached to the mask body.

Clause 41. The ventilation mask of Clause 18-40, wherein the hub housing is sealingly engaged to the mask body.

Clause 42. The ventilation mask of Clause 18-41, wherein the drug delivery hub can be removably coupled to the mask body such that the ventilation mask can provide a gas to the patient cavity when the drug delivery hub is coupled to the mask body and when the drug delivery hub is uncoupled from the mask body.

Clause 43. A ventilation mask, comprising: a mask body defining a patient cavity, the mask body comprising a patient opening in fluid communication with the patient cavity; a therapeutic gas manifold coupled to the mask body, the therapeutic gas manifold defining a therapeutic gas channel, the therapeutic gas manifold comprising a plurality OF therapeutic gas ports in fluid communication with the therapeutic gas channel, wherein the plurality of therapeutic gas ports are configured to create a therapeutic gas flow within the patient cavity; and a drug delivery hub, comprising: a hub housing defining a housing volume, wherein the hub housing is disposed between the mask body and the therapeutic gas manifold; a reservoir disposed at least partially within the housing volume, wherein the reservoir comprises a reservoir volume configured to dispense a medicament; a nebulizer channel in fluid communication with the reservoir volume and configured to be in fluid communication with the patient cavity; and a gas channel in fluid communication with the nebulizer channel, wherein the gas channel is configured to direct a gas flow toward the patient cavity through the nebulizer channel, drawing a portion of the medicament from the reservoir through the nebulizer channel with the gas flow toward the patient cavity.

Clause 44. The ventilation mask of Clause 43, wherein the gas channel and the therapeutic gas channel are in fluid communication with a gas supply line.

Clause 45. The ventilation mask of Clause 43 or 44, wherein the gas channel receives the gas flow from the therapeutic gas manifold.

Clause 46. The ventilation mask of Clause 43-45, further comprising a fill port defined in the mask body, wherein the fill port is in fluid communication with the reservoir to permit access to the reservoir volume.

Clause 47. The ventilation mask of Clause 43-46, wherein the reservoir is disposed toward an upper portion of the hub housing.

Clause 48. The ventilation mask of Clause 43, wherein the reservoir is disposed toward a lower portion of the hub housing.

Clause 49. The ventilation mask of Clause 43, wherein the reservoir is disposed toward a lateral portion of the hub housing.

Clause 50. The ventilation mask of Clause 43, wherein the reservoir is removably attached to the hub housing.

Clause 51. The ventilation mask of Clause 50, wherein the reservoir is removably attached to the housing volume.

Clause 52. The ventilation mask of Clause 43, wherein the medicament comprises a liquid medicament.

Clause 53. The ventilation mask of Clause 43, wherein the medicament comprises a powdered medicament.

Clause 54. A method comprising: introducing a therapeutic gas into a patient cavity of a ventilation mask via a plurality of gas ports; directing a gas flow through a nebulizer channel of a drug delivery hub coupled with the ventilation mask; drawing a portion of a medicament from a reservoir through the nebulizer channel with the gas flow, wherein the reservoir is disposed within the drug delivery hub; and directing the gas flow and the portion of the medicament into the patient cavity of the ventilation mask.

Clause 55. The method of Clause 54, further comprising attaching the drug delivery hub to the ventilation mask.

Clause 56. The method of Clause 55, wherein attaching the drug delivery hub comprises attaching the drug delivery hub to a vent opening of the ventilation mask.

Clause 57. The method of Clause 54-56, further comprising removing the drug delivery hub from the ventilation mask.

Clause 58. The method of Clause 54-57, further comprising introducing additional medicament into the reservoir.

Clause 59. The method of Clause 54-58, further comprising providing the gas flow to the nebulizer channel via a gas supply line.

Clause 60. The method of Clause 54-59, further comprising providing the gas flow to the nebulizer channel and the ventilation mask via a gas supply line.

Clause 61. The method of Clause 54-60, further comprising providing the gas flow to the nebulizer channel via at least one of the plurality of gas ports.

Clause 62. The method of Clause 54-61, further comprising providing the gas flow to the nebulizer channel an access port formed through the ventilation mask.

Clause 63. The method of Clause 54-62, further comprising directing the gas flow through the nebulizer channel to create negative pressure to draw the portion of the medicament from the reservoir.

Clause 64. The method of Clause 54-63, further comprising pulverizing the portion of the medicament into an aerosol in the nebulizer channel.

Clause 65. The method of Clause 54-64, further comprising introducing the reservoir into the drug delivery hub.

Clause 66. The method of Clause 54-65, further comprising removably attaching the reservoir to the drug delivery hub.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A drug delivery hub, comprising:
a hub housing defining a housing volume, wherein the hub housing is configured to directly couple with a mask defining a patient cavity;
a reservoir disposed at least partially within the housing volume, wherein the reservoir comprises a reservoir volume configured to dispense a medicament;
a nebulizer channel in fluid communication with the reservoir volume and configured to be in fluid communication with the patient cavity when the hub is coupled with the mask; and
a gas channel in fluid communication with the nebulizer channel, wherein the gas channel is configured to direct a gas flow toward the patient cavity through the nebulizer channel, drawing a portion of the medicament from the reservoir through the nebulizer channel with the gas flow toward the patient cavity.

2. The drug delivery hub of claim 1, wherein the gas channel comprises an access port to receive the gas flow.

3. The drug delivery hub of claim 2, wherein the access port is configured to engage with a self-sealing membrane or luer connection.

4. The drug delivery hub of claim 1, wherein the hub housing comprises an inner surface that defines a passage through the hub, such that, when the hub is coupled with the mask, the passage extends through the hub to the patient cavity.

5. The drug delivery hub of claim 1, wherein, when the hub is coupled with a mask, the hub is configured to receive a gas from the mask and direct the gas through the hub to the patient cavity.

6. The drug delivery hub of claim 1, wherein, when the hub is coupled with a mask, the hub is configured to receive a gas from the mask and direct the gas through the hub and back to the mask.

7. The drug delivery hub of claim 1, wherein the hub is configured to be removably coupled to a mask.

8. A ventilation mask, comprising:
a mask body defining a patient cavity, the mask body comprising a patient opening in fluid communication with the patient cavity; and
a drug delivery hub, comprising:
a hub housing defining a housing volume, wherein the hub housing is directly coupled to the mask body;
a reservoir disposed at least partially within the housing volume, wherein the reservoir comprises a reservoir volume configured to dispense a medicament;
a nebulizer channel in fluid communication with the reservoir volume and configured to be in fluid communication with the patient cavity; and
a gas channel in fluid communication with the nebulizer channel, wherein the gas channel is configured to direct a gas flow toward the patient cavity through the nebulizer channel, drawing a portion of the medicament from the reservoir through the nebulizer channel with the gas flow toward the patient cavity.

9. The ventilation mask of claim 8, wherein the drug delivery hub can be removably coupled to the mask body such that the ventilation mask can provide a gas to the patient cavity when the drug delivery hub is coupled to the mask body and when the drug delivery hub is uncoupled from the mask body.

10. The ventilation mask of claim 8, further comprising:
at least one vent opening formed through the mask body, the at least one vent opening in fluid communication with the patient cavity, wherein the at least one vent opening is disposed generally opposite to the patient opening, and the hub housing is coupled to the at least one vent opening.

11. The ventilation mask of claim 10, wherein the hub housing comprises a continuous surface and at least partially obstructs the at least one vent opening.

12. The ventilation mask of claim 10, wherein the hub housing comprises an inner surface that defines a passage through the hub to the patient cavity.

13. The ventilation mask of claim 8, further comprising:
a therapeutic gas manifold coupled to the mask body, the therapeutic gas manifold defining a therapeutic gas channel, the therapeutic gas manifold comprising a plurality of therapeutic gas ports in fluid communication with the therapeutic gas channel, wherein the plurality of therapeutic gas ports are configured to create a therapeutic gas flow within the patient cavity.

14. The ventilation mask of claim 13, wherein the gas channel and the therapeutic gas channel are in fluid communication with a gas supply line.

15. The ventilation mask of claim 13, wherein the gas channel receives the gas flow from the therapeutic gas manifold.

16. The ventilation mask of claim 15, wherein the gas channel is in fluid communication with at least one of the plurality of therapeutic gas ports.

17. The ventilation mask of claim 13, wherein, when the hub is coupled with a mask, a portion of the hub obstructs at least a portion of the plurality of therapeutic gas ports to scavenge a gas exiting therefrom and direct the scavenged gas into the gas channel of the hub.

18. A method comprising:
introducing a therapeutic gas into a patient cavity of a ventilation mask via a plurality of gas ports;
directing a gas flow through a nebulizer channel of a drug delivery hub coupled with the ventilation mask;
drawing a portion of a medicament from a reservoir through the nebulizer channel with the gas flow, wherein the reservoir is disposed within the drug delivery hub; and
directing the gas flow and the portion of the medicament into the patient cavity of the ventilation mask.

19. The method of claim 18, further comprising attaching the drug delivery hub to the ventilation mask.

20. The method of claim 19, wherein attaching the drug delivery hub comprises attaching the drug delivery hub to a vent opening of the ventilation mask.

21. The method of claim 18, further comprising removing the drug delivery hub from the ventilation mask.

22. The method of claim 18, further comprising introducing additional medicament into the reservoir.
